(19) United States
(12) Patent Application Publication
(10) Pub. No.: US 2021/0198182 A1
(43) Pub. Date: Jul. 1, 2021

(54) ESTERS OF AMINO CARBOXYLIC ACIDS AND A PROCESS TO PREPARE THEM

(71) Applicant: NOURYON CHEMICALS INTERNATIONAL B.V., ARNHEM (NL)

(72) Inventors: Natalija SMOLKO SCHWARZMAYR, Deventer (NL); Ruifei WANG, Deventer (NL); Alexander Michael WETZEL, Deventer (NL); Louis Eckhard SCHWARZMAYR, Deventer (NL); Göran Thomas LJUNGDAHL, Deventer (NL); Krzysztof Piotr KOLMAN, Deventer (NL)

(73) Assignee: NOURYON CHEMICALS INTERNATIONAL B.V., ARNHEM (NL)

(21) Appl. No.: 17/247,756

(22) Filed: Dec. 22, 2020

(30) Foreign Application Priority Data
Dec. 30, 2019 (EP) 19220141.6

(51) Int. Cl. C07C 227/02; C07C 227/22

(52) U.S. Cl. CPC C07C 227/02; C07C 229/06; C07C 227/22

(57) ABSTRACT

A process to prepare an amine-functional ester of an amino carboxylic acid includes the step of reacting a polyol of the formula (I)

HO-A-OH (I)

where A is a carbon chain with about 2 to about 36 carbon atoms that is aliphatic linear or branched, saturated or unsaturated, or aromatic, or CH₂CH(OH)CH₂, CH₂C(CH₂OH)₂CH₂, CH₂C(CH₂OH)(CH₃)CH₂, CH₂C(CH₂OH)(CH₂CH₃)CH₂, p-tetrahydrofuran, erythritol or an ester of di- or tricarboxylic acids with ethylene or propylene glycol, and wherein A can optionally be alkoxylated or reacted with hydroxy carboxylic acids, with an aminocarboxylic acid of formula II or its cyclic amide of the formula III, where m is an integer of about 1 to about 8 in formula II and about 3 to about 8 in formula III, each R independently is hydrogen or a C1-C4 alkyl group, or CH₂CH₂COOH, or CH₂COOH, or (CH₂)₄NH₂, in the presence of a Brønsted-Lowry acid.

### CROSS-REFERENCE TO RELATED APPLICATION

This application claims priority to European Application No. 19220141.6, filed Dec. 30, 2019, which is hereby incorporated by reference in its entirety.

### TECHNICAL FIELD

The present disclosure relates to a process to prepare amine-functional esters of amino carboxylic acids and polyols and to the amine-functional esters obtainable by such process.

### BACKGROUND

In a number of documents the reaction of a monoalcohol with an amino carboxylic acid is disclosed.

For example, a process to prepare esters of aminocarboxylic acids is disclosed in “Esters of 6-aminohexanoic acid as skin permeation enhancers: The effect of branching in the alkanol moiety”, A Habralek et al, Journal of Pharmaceutical Sciences, Vol. 94, 1494-1499, (2005). The process disclosed in this document involves in a first step a conversion of the amino carboxylic acid to an amino acyl chloride that next reacts with an alkanol to give an ammonium functional ester that next is deprotonated to give the aminocarboxylic acid ester.

JP S49-082624 discloses a process to prepare esters of aminocarboxylic acids in which an aminocarboxylic acid or a cyclic amide thereof is first reacted with a mineral acid catalyst which will convert this aminocarboxylic acid or cyclic amide into its aminocarboxylic acid salt, and next this aminocarboxylic acid salt is esterified with an alkanol to give the ester of the alkanol and the aminocarboxylic acid. This process is hence an esterification (or condensation) reaction, in embodiments wherein the cyclic amide precursor is employed, preceded by a de-esterification (or hydrolysis) reaction. The process is done at a temperature of 150 deg C. and the alkanol reactant is 20% overdosed on cyclic amide molar amount.

J Klimentova, et al, in “Transkarbams with terminal branching as transdermal permeation enhancers”, Bioorganic & Medicinal Chemistry Letter 18 (2008), 1712-1715 disclose also an esterification reaction of the salt of an aminocarboxylic acid with an alkanol in the presence of thionylchloride. The molar ratio of aminocarboxylic acid salt to alkanol in this document is 6.16:5.54 which corresponds with 1.11:1.00.

U.S. Pat. No. 4,216,227 discloses a process to prepare omega amino acid esters by reacting omega amino acid salts with an excess of alkanol in the presence of dry HCl gas. The details of the process are not disclosed, other than that a temperature of 150 deg C. is employed for 3 hours, and neither is the use of cyclic amide instead of amino acid salt disclosed.

EP 847987 also discloses a process to prepare esters of aminocarboxylic esters. The process disclosed in EP 987 involves reacting an aminocarboxylic acid or a cyclic amide thereof with an alkanol in the presence of a divalent or trivalent inorganic acid, wherein the alkanol is dosed in high molar excess relative to the amount of the aminocarboxylic acid and later distilled off. The process is said to be performed under reflux conditions for several hours.

U.S. Pat. No. 3,211,781, like EP 847987, discloses a process to prepare esters of amino carboxylic acids wherein a cyclic amide of an amino carboxylic acid is reacted with an alkanol in the presence of a Brønsted-Lowry acid under reflux conditions. Also, in this document the alkanol is dosed in a high molar excess compared to the molar amount of cyclic amide.

Lele, Gore and Kulkarni in Direct Esterification of Poly)ethylene glycol) with amino acid hdyrochlorides, Synthetic Communications, 29:10, 1727-1739 (1999) disclose a process of reacting polyethyleneglycol PEG 6000 with an amino acid in the HCl form in the presence of dicyclohexyl carbodiimide

U.S. Pat. No. 3,939,200 discloses a process of reacting an amino acid in its salt form with a diol wherein the diol is an alkane or cycloalkane diol. The amino acids are first converted to their, preferably HCl, salt. It is indicated that when a cyclic amide is employed it will have to be first hydrolyzed to ring open the structure. In the Examples solvents such as trichloropropane or toluene are used in a quite high amount. Halogenated alkanes and aromatics are undesirable solvents as they have HSE issues, such as suspected carcinogenity.

### BRIEF SUMMARY

This disclosure provides a process to prepare amine-functional esters of an amino carboxylic acid comprising the steps of reacting a polyol of the formula (I)

HO-A-OH (I)
where A is a carbon chain with about 2 to about 36 carbon atoms that is aliphatic linear or branched, saturated or unsaturated, or aromatic, or CH₂CH(OH)CH₂, CH₂C(CH₂OH)₂CH₂, CH₂C(CH₂OH)(CH₃)CH₂, CH₂C(CH₂OH)(CH₂CH₃)CH₂, p-tetrahydrofuran, erythritol or an ester of di- or tricarboxylic acids with ethylene or propylene glycol, and wherein A can optionally be alkoxylated or reacted with hydroxy carboxylic acids, with an aminocarboxylic acid of formula II or its cyclic amide of the formula III

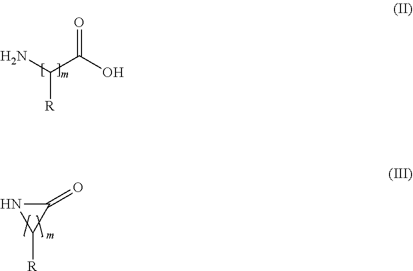

where m is an integer of 1 to 8 in formula II and 3 to 8 in formula III, each R independently is hydrogen or a C1-C4 alkyl group, or CH₂CH₂COOH, or CH₂COOH, or (CH₂)₄NH₂
in the presence of a Brønsted-Lowry acid at a temperature of between about 60 and about 200 degrees C. wherein the total molar amount of aminocarboxylic acid or its cyclic amide to the molar amount of the polyol is from about n:0.8 to about n:1.5 wherein n is the total number of hydroxyl groups on the polyol and wherein the Brønsted-Lowry acid is not added to the reaction mixture until at least about 50% of the total of the polyol and the aminocarboxylic acid or its cyclic amide are dosed.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has been found that a process to prepare amine-functional esters of aminocarboxylic acids and polyols can be performed by dosing the polyol and (the cyclic amide of) aminocarboxylic acid in the right molar ratios of about n moles of aminocarboxylic on each mole polyol containing n hydroxyl groups, so that the polyol does not have to be distilled off and furthermore that the (trans)esterification reaction when performing the process in this way can be catalyzed by many Brønsted-Lowry acids in the absence of solvent or using a limited amount of solvent.

The present disclosure now provides an improved process to prepare esters of an amino carboxylic acid comprising the steps of reacting a polyol of the formula (I) HO-A-OH (I) where A is a carbon chain with about 2 to about 36 carbon atoms that can be aliphatic (linear or branched, saturated or unsaturated) or aromatic, or CH₂CH(OH)CH₂, CH₂C(CH₂OH)₂CH₂, CH₂C(CH₂OH)(CH₃)CH₂, CH₂C(CH₂OH)(CH₂CH₃)CH₂, p-tetrahydrofuran, erythritol or an ester of di- or tricarboxylic acids with ethylene or propylene glycol, and wherein A can optionally be alkoxylated with one or more alkylene oxide units or reacted with one or more hydroxy carboxylic acids,

with an aminocarboxylic acid of formula II or its cyclic amide of the formula III

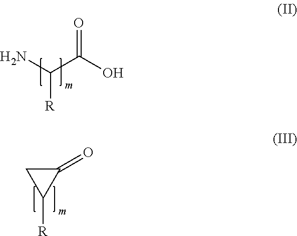

where m is an integer of 1-8 in formula II and 3-8 in formula III, each R independently is hydrogen or a C1-C4 alkyl group, or CH₂CH₂COOH, or CH₂COOH, or (CH₂)₄NH₂ in the presence of a Brønsted-Lowry acid at a temperature of between about 60 and about 200 degrees C. wherein the total molar amount of aminocarboxylic acid or its cyclic amide to the molar amount of the polyol is between about n:0.8 and about n:1.5 wherein n is the total number of hydroxyl groups on the polyol and wherein the Brønsted-Lowry acid is not added to the reaction mixture until at least about 50% of the total of the polyol and the aminocarboxylic acid or its cyclic amide are dosed.

The disclosure furthermore provides amine-functional esters of amino carboxylic acids as obtainable by the process of the disclosure.

The compounds obtainable by the process of the disclosure can in many embodiments be covered by the below structure

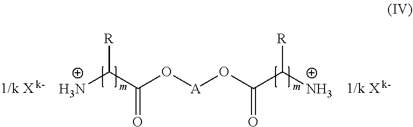

wherein k is a value of 1 to 3, each R independently is as defined above, A is as defined above, each m is as defined above, and X is an anion derivable from deprotonating a Brønsted-Lowry acid.

If following the above options for A, in the cases wherein the group A contains further hydroxyl substituents these may converted into groups wherein the proton of the OH group is substituted by the group of formula (V)

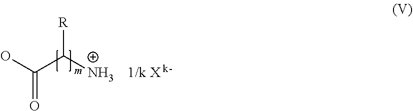

wherein k, each R independently, m and X are as defined above.

The compounds obtainable by the process of the disclosure may contain between about 0 and about 30 mole % on total moles of compounds (IV) of compounds below formula (VI)

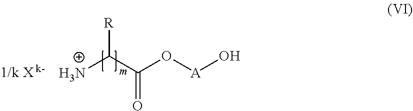

wherein k, each R independently, A, m and X are as defined above. Preferably the amount of compounds of formula VI is between about 1 and about 20 mole %, even more typically between about 2 and about 15 mole %, typically about 5 and about 10 mole %, on total amount of compound IV.

The polyols that can be used in the process are compounds containing at least two hydroxyl groups of the formula HO-A-OH (I) where A is a carbon chain with about 2 to about 36 carbon atoms that can be aliphatic (linear or branched, saturated or unsaturated) or aromatic, or CH₂CH(OH)CH₂, CH₂C(CH₂OH)₂ CH₂, CH₂C(CH₂ OH)(CH₃)CH₂, CH₂C(CH₂ OH)(CH₂CH₃)CH₂, p-tetrahydrofuran, erythritol or esters of di(tri)carboxylic acids with ethylene or propylene glycol, optionally alkoxylated with one or more alkylene oxide unit or reacted with one or more hydroxy carboxylic acids.

Examples of polyols that are alkoxylated are HO—(CH₂—CH(R1)-O)p-CH₂—CH(R1)-(O—CH₂—CH(R1))p-OH, HO—(CH₂—CH(R1)-O)p-CH₂—CH═CH—CH₂—(O—CH₂—CH(R1))p-OH, HO—(CH₂—CH(R1)-O)p-CH₂—CH(CH₃)₂—CH₂—(O—CH₂—CH(R1))p-OH, HO—(CH₂—CH(R1)-O)p-C₆H₄—(O—CH₂—CH(R1))p-OH, HO—(CH₂—CH(R1)-O)p-CH₂—C₆H₄—CH₂—(O—CH₂—CH(R1))p-OH, HO—(CH₂—CH(R1)-O)p-C₆H₁₀—(O—CH₂—CH(R1))p-OH, HO—(CH₂—CH(R1)-O)p-CH₂—C₆H₁₀—CH₂—(O—CH₂—CH(R1))p-OH, HO—(CH₂—CH(R1)-O)p-CH₂—(CH₂)n-CH₂—(O—CH₂—CH(R1))p-OH, HO—(CH₂—CH(R1)-O)p-CH₂—CH₂—CH₂—CH₂—(O—CH₂—CH₂—CH₂—CH₂)p-(O—CH₂—CH(R1))p-OH, HO—(CH₂—CH(R1)-O)p-(CH₂)i-CH((CH₂)jCH₃)—CH((CH₂)jCH₃)—(CH₂)i-(O—CH₂—CH(R1))p-OH, HO—(CH₂—CH(R1)-O)p-CH₂—(CH(O—CH₂—CH(R1))p-OH))d-CH₂—(O—CH₂—CH(R1))p-OH, HO—(CH₂—CH(R1)-O)p-CH₂—C(CH₂—(O—CH₂—CH(R1))p-OH)₂—CH₂—(O—CH₂—CH(R1))p-OH, HO—(CH₂—CH(R1)-O)p-CH₂—C(CH₂—(O—CH₂—CH(R1))p-OH)(CH₃)—CH₂—(O—CH₂—CH(R1))p-OH, HO—(CH₂—CH(R1)-O)p-CH₂—C(CH₂—(O—CH₂—CH(R1))p-OH)(CH₂CH₃)—CH₂—(O—CH₂—CH(R1))p-OH, wherein each p is independently an integer of from about 0-25, as long as one p in a structure is at least about 1, n is 1-10, i+j=16, d=1-4 and each R1 independently is hydrogen atom or C1-C2 alkyl group.

Examples of polyols that are alcohols reacted with hydroxy carboxylic acids are compounds of below formulae: HO—(CH(Z)—C(O)—O)p-CH₂—CH(R1)-(O—C(O)—CH(Z))p-OH, HO—(CH(Z)—C(O)—O)p-CH₂—CH═CH—CH₂—(O—C(O)—CH(Z))p-OH, HO—(CH(Z)—C(O)—O)p-CH₂—CH(CH₃)₂—CH₂—(O—C(O)—CH(Z))p-OH, HO—(CH(Z)—C(O)—O)p-C6H₄-(O—C(O)—CH(Z))p-OH, HO—(CH(Z)—C(O)—O)p-CH₂—C6H₄—CH₂—(O—C(O)—CH(Z))p-OH, HO—(CH(Z)—C(O)—O)p-C6H₁₀-(O—C(O)—CH(Z))p-OH, HO—(CH(Z)—C(O)—O)p-CH₂—C₆H₁₀—CH₂—(O—C(O)—CH(Z))p-OH, HO—(CH(Z)—C(O)—O)p-CH₂—(CH₂)n-CH₂—(O—C(O)—CH(Z))p-OH, HO—(CH(Z)—C(O)—O)p-CH₂—CH₂—CH₂—CH₂—(O—CH₂—CH₂—CH₂—CH₂)p-(O—C(O)—CH(Z))p-OH, HO—(CH(Z)—C(O)—O)p-(CH₂)i-CH((CH₂)jCH₃)—CH((CH₂)jCH₃)—(CH₂)i-(O—C(O)—CH(Z))p-OH, HO—(CH(Z)—C(O)—O)p-CH₂—(CH(O—C(O)—CH(Z))p-OH))d-CH₂—(O—C(O)—CH(Z))p-OH, HO—(CH(Z)—C(O)—O)p-CH₂—C(CH₂—(O—C(O)—CH(Z))p-OH)₂—CH₂—(O—C(O)—CH(Z))p-OH, HO—(CH(Z)—C(O)—O)p-CH₂—C(CH₂—(O—C(O)—CH(Z))p-OH)(CH₃)—CH₂—(O—C(O)—CH(Z))p-OH, HO—(CH(Z)—C(O)—O)p-CH₂—C(CH₂—(O—C(O)—CH(Z))p-OH)(CH₂CH₃)—CH₂—(O—C(O)—CH(Z))p-OH, HO—(CH(Z)—C(O)—O)p-CH₂(CH₂)k-CH(R1)-(O—C(O)—CH(Z))p-OH, HO(CH(Z)—C(O)—O)p-CH₂—(CH(O—C(O)—CH(Z)—OH)p)e-CH₂—(O—C(O)—CH(Z))pOH

wherein each p is independently an integer of from about 0-25, as long as at least one p in a structure is at least about 1, and each R1 independently is hydrogen atom or C1-C2 alkyl group, k is about 0-10, if k>0 then R1 is hydrogen, Z is hydrogen or CH₃, e=1-4.

Examples of polyols that are esters of di- or tricarboxylic acids with ethylene glycol or propylene glycol are compounds of below formula HO(CH₂CH(R1)-O)p-C(O)—CH₂—(C(OH)(C(O)(O—CH(R1)-CH₂)pOH)d-(CH₂)k-C(O)(O—CH(R1)-CH₂)pOH

wherein each p is independently an integer of from about 0-25 as long as at least one p in a structure is at least about 1, and each R1 independently is hydrogen atom or C1-C2 alkyl group, d=0-1, when d=1, then k=1; when d=0, then k=0-10

Most preferred polyols are chosen from ethylene glycol, propylene glycol, butylene glycol, polyethylene glycol, polypropylene glycol, polybutylene glycol, glycerol, poly tetrahydrofuran, pentaerythritol, trimethylolpropane, trimethylolethane, diethylenesuccinate, dimeric fatty alcohol, 1,6-hexandiol, 1,4-butandiol, 1,8-octandiol, 1,10-decandiol, erythritol, optionally alkoxylated or reacted with hydroxy carboxylic acid. When using polyethylene glycol, polypropylene glycol or polybutylene glycol these preferably contain about 2 to 10 ethyleneoxide, propyleneoxide, resp butyleneoxide units, typically about 2 to 6 units.

Mixtures of two or more polyols may also be employed.

Preferred aminocarboxylic acids of formula II are glycine, alanine, valine, leucine, isoleucine, lysine, aspartic acid, and glutamic acid, more preferred are glycine or alanine. Further preferred aminocarboxylic acids of formula II are chosen from 6-aminohexanoic acid, 4-aminobutanoic acid.

In a preferred embodiment a cyclic amide of formula III is employed in the process of the disclosure. It is more preferred that in formula III m is 3, 4, 5 or 6. Most preferred are the cyclic amides of 6-aminohexanoic acid, 4-aminobutanoic acid.

It should be noted that a mixture of aminocarboxylic acids and/or of cyclic amides can also be employed in the process.

Preferred amine-functional esters of formula (IV) include

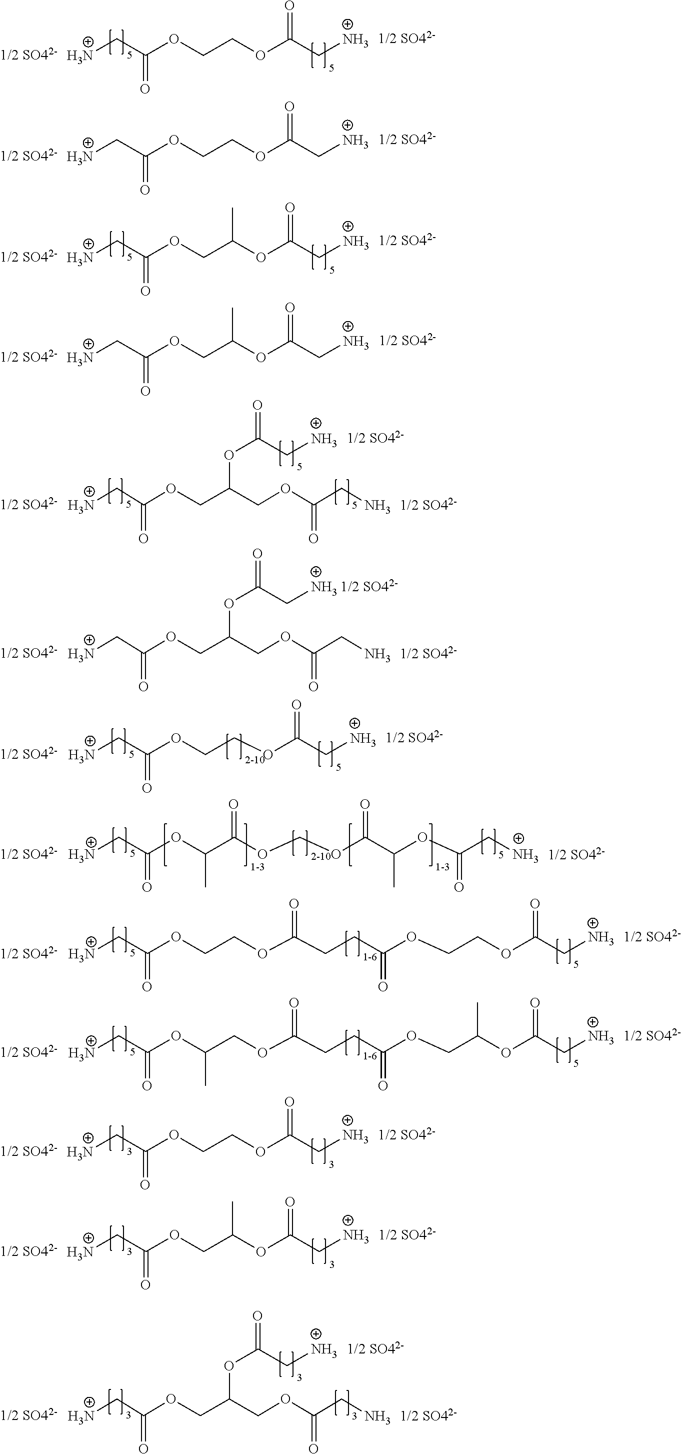

The compounds of formula (IV), (VI) were determined to have an improved biodegradability and/or chemical degradability compared to many commercially available amine-functional compounds that contain at least two amine groups, which adds to the environmental profile of any application in which they are used. They can be employed as alternatives with better degradability profile in applications wherein diamines/polyamines are presently used such as in the preparation of polymers that contain diamine monomers. Such polymers include polyurea, polyepoxide, and polyamide polymers and there is a desire to make such polymers biodegradable. Furthermore, they can be employed as building blocks wherein the amine is further modified to make imide- or (meth)acrylamide-functional monomers.

In preferred embodiments the compounds of formula (IV), (VI) are readily biodegradable.

In a preferred embodiment of the process of the disclosure the aminocarboxylic acid is used in its cyclic amide form (of formula III). The advantage of performing a transesterification reaction of a cyclic amide of an amino carboxylic acid instead of an esterification of an amino carboxylic acid or salt thereof is that it is much easier to control the amount of water in the process. When doing a transesterification reaction there is no net formation of water. When performing an esterification reaction, more water will form and such increased water content may give rise to side reactions, such as in particular under an acidic pH, a hydrolysis reaction wherein formed product falls apart in the starting compounds again. Also, when attempting to remove water from the reaction mixture, the reactants, most notably the polyols, might form an azeotrope with the water and get lost as well.

Furthermore, it has been found that the side reaction wherein the cyclic amide self-polymerizes that is suppressed in state of the art processes by diluting the reaction mixture with an excess of alkanol reactant was found to be similarly suppressed in the process of the disclosure, wherein the reactants cyclic amide and polyol are dosed such that around about 1 mole of cyclic amide is present per hydroxyl group on the polyol, especially in embodiments wherein the process is done under somewhat more moderate temperatures, such as temperatures under about 150 deg C. Quite unexpectedly, in the process of the disclosure the (trans)esterification reaction takes similarly long as in the prior art processes wherein the alkanol is clearly overdosed and still very similar yields of desired product are obtained. Finally, it is an advantage that the excess of polyol does not need to be removed from the reaction mixture.

In the process of the disclosure the amino carboxylic acid or its cyclic amide derivative, and the polyol are preferably employed in a molar ratio of total aminocarboxylic acid and/or cyclic amide to polyol that is preferably between about n:0.8 and about n:1.3, more preferably between about n:0.9 and about n:1.1, wherein n stands for the number of hydroxyl groups on the polyol, most preferably both compounds are used in a substantially equimolar amount of about 1 mole of aminocarboxylic acid per hydroxyl group.

Unexpectedly the reaction can be performed under relatively mild conditions, such as a milder reaction temperature, and provide high yields of the ester if the polyol is used in the ratio of the present disclosure. Effectively the reaction was found to progress substantially towards the claimed ester product in which there is only a limited remaining amount of unreacted polyol and aminocarboxylic acid or cyclic amide and in which only trace amounts of only one side product that is the reaction product of polyol and Brønsted-Lowry acid are present.

In applications wherein a considerable amount of polyol or side products caused by not being able to control the amount of water, create a problem but a minor amount of unreacted polyol or aminocarboxylic acid or its cyclic amide are not an issue, the product of the process of the disclosure can be used without intermediate steps to remove impurities such as for example unreacted starting materials.

The Brønsted-Lowry acid is preferably not added to the reaction mixture until at least about 50% of the total of the polyol and at least about 50% of the total of the (cyclic amide of the) aminocarboxylic acid are added. In a preferred embodiment at least about 75% of the total of the polyol and at least about 75% of the aminocarboxylic acid or its cyclic amide are dosed before the Brønsted-Lowry acid is added to the reaction mixture, even more preferably at least about 90%. In a preferred embodiment, at least part of the polyol is first dosed to the reactor and next at least part of the aminocarboxylic acid or its cyclic amide, after which the Brønsted-Lowry acid is added. It however is also possible to dose the aminocarboxylic acid or its cyclic amide partially or completely before the polyol is dosed to the reactor or to simultaneously dose the cyclic amide and the polyol after which the Brønsted-Lowry acid is added.

The temperature is preferably increased to at least about 60 deg C. and to up to about 200 deg C. after the reactants and the Brønsted-Lowry acid have been dosed. It is beneficial to first increase the temperature to between about 60 and about 100 deg C. and next to a temperature between about 100 and 150 deg C., or typically about 110 and about 140 deg C.

The process can be performed in a solvent or without a solvent. If a solvent is used it is preferably an organic solvent that is not an aromatic solvent, not a halogenated alkane solvent and not an alcoholic solvent. Solvents that are suitable to use in the process of the disclosure can be chosen from alkane and ether solvents with a boiling point higher than about 80 deg C. more preferably higher than about 100 deg C., even more preferably higher than about 120 deg C. Water can also be used. The process is preferably performed with an amount of solvent that is about 0 to about 50 wt %, typically about 0 to about 25 wt %, about 0-10 wt %, based on total reactants.

In a preferred embodiment during the process the molar amount of water on total moles of polyol is between about 0 and about 10 mole %, more typically about 0.01 and about 5 mole %, most typically about 0.1 and about 2 mole %.

After the reaction is completed, optionally (to decrease viscosity) the product can be diluted with water and/or an organic polar solvent. The preferred organic solvents are glycolic solvents, such as propylene glycol, triethylene glycol, ethylene glycol, 2-methoxyethanol, glycerol, or isopropanol.

The Brønsted-Lowry acid is preferably used as a concentrated (i.e. about 20 to about 100 wt %) aqueous solution and dosed to the reaction mixture containing all the aminocarboxylic acid or its cyclic amide and the polyol in portions to control the exothermal effect of the reaction.

The Brønsted-Lowry acid is not an aminocarboxylic acid. The Brønsted-Lowry acid is preferably an acid with a pKa of between about −10 and about 3. The Brønsted-Lowry acid is even more preferably an inorganic acid, even more preferably it is sulfuric acid, phosphoric acid or a hydrohalic acid and most preferably it is sulfuric acid as hydrohalic acids such as HCl in embodiments result in solid products and phosphoric acid results in lower conversions. Moreover, both HCl and H3PO4 as a liquid are available as solutions with quite some water (HCl 37% conc, H3PO4 85% conc) while H2SO4 is available in substantially water-free form (such as about 95-98% concentrate). The use of H2SO4 therefore allows to get products of high conversion (>about 90%) in a liquid form, and moreover no need to evaporate water exists.

X is in a preferred embodiment an anion derived from an inorganic Brønsted-Lowry acid, more preferred a halogenide, sulphate, hydrogen sulfate, hydrogen phosphate, dihydrogen phosphate, or phosphate anion. Most preferably, X is a sulfate or hydrogen sulfate anion.

The process can be done under reduced pressure but is preferably performed at a pressure that is atmospheric.

The product can optionally be neutralized by a base to a pH from about 3-7. The product can be purified by methods available to someone skilled in the art. However, because of the low level of side products, it can also be used without further processing or purification steps, such as a surfactant.

The process is very favorable for polyols that have a boiling point up to about 220 deg C., or, for polyols that can form an azeotrope with water having a boiling point lower than about 220 deg C. Compared to the state of the art processes, in the process of the disclosure when using such polyols, they will not be stripped from the reaction mixture when reaction water is removed, as in the process of the disclosure no such water is formed and hence does not need to be removed. Many polyols that can be used in the process of the disclosure have a boiling point in this range (especially the polyols that are smaller or that contain some branching in their structure).

The process is preferably done at a temperature of between about 60 and about 150 deg C., more typically between about 80 and about 145 deg C., most typically about 110 and about 140 deg C.

### EXAMPLES

Compounds used

### ε-Caprolactam (ex Acros Organics)

pyrrolidone (ex Acros Organics)
Monoethylene glycol (ex Sigma-Aldrich)

### Polytetrahydrofurane (ex Sigma-Aldrich)

triethyleneglycol (ex Sigma-Aldrich)
glycerol (ex Sigma-Aldrich)

### H2SO4 (95%) (ex VWR)

### Example 1—Preparation of ethane-1,2-diyl bis(6-aminohexanoate) sulphate Salt

A 250 mL flanged glass reactor with overhead stirrer, thermometer and cooler was loaded with ε-caprolactam (89.9 g, 0.79 mol) and ethylene glycol (24.6 g, 0.4 mol). The mixture was heated to 80 deg C. Then, sulphuric acid (95% in water 55.94 g, 0.54 mol) was added and the mixture was heated to 140 deg C. After 8 hours, the mixture was cooled to room temperature and analysed by NMR. The product was obtained as a viscous liquid. The product contained ethane-1,2-diyl bis(6-aminohexanoate) salt in a high yield of more than 80% and a small amount of hydroxyethyl-6-aminohexanoate sulphate salt.

### Example 2—Preparation of Diesterdiamine from Triethyleneglycol and Caprolactam

Caprolactam (90.2 g, 797 mmol) and triethylene glycol (59.8 g, 399 mmol) were charged in a 500 mL flanged glass reactor and heated to 60° C. (externally measured temperature) using mechanical stirring, under a stream of nitrogen. Sulfuric acid (60 g, 578 g) was added over 25 mins and the temperature was then increased to 135° C. and the reaction was stopped after approximately 13 h. The product was isolated and analyzed by ¹H NMR.

### Example 3—Preparation of Diesterdiamine from Poly-THF and Caprolactam

Caprolactam (16.9 g, 149 mmol) and Terathane 1400 (102.6 g, 75 mmol) were charged in a 1 L flanged glass reactor and heated to 60° C. (externally measured temperature) using mechanical stirring, under a stream of nitrogen. Sulfuric acid (11 g, 108 mmol) was added over 25 mins and the temperature was then increased to 135° C. and the mixture was further stirred for approximately 19 h. The product was isolated and analyzed by ¹H NMR. Approximately 70% yield was established.

### Example 4—Preparation of Triestertriamine from Glycerol and Caprolactam

Caprolactam (212 g, 1880 mmol) and glycerol (57.6 g, 625 mmol) were charged in a 500 mL flanged glass reactor and heated to 60° C. (externally measured) using mechanical stirring, under a stream of nitrogen. Sulfuric acid (118 g, 1100 mmol) was added over 45 mins and the temperature was then increased to 135° C. and the mixture was further stirred for approximately 14 h. The product was isolated and analyzed by ¹H NMR. The product contained propane-1,2,3-triyl tris(6-aminohexanoate) salt in a high yield of more than 80%.

### Example 5—Preparation of Diesterdiamine from Ethylene Glycol and 2-pyrrolidinone

2-pyrrolidinone (93.7 g, 1100 mmol) and ethylene glycol (34.5 g, 556 mmol) were charged in a 1 L flanged glass reactor and heated to 60° C. (externally measured temperature) using mechanical stirring, under a stream of nitrogen. Sulfuric acid (82 g, 790 mmol) was added over 45 mins and the temperature was then increased to 135° C. and the mixture was further stirred for approximately 25 h. The product was isolated and analyzed by ¹H NMR. Approximately 80% yield was established.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. Process to prepare amine-functional esters of an amino carboxylic acid comprising the steps of reacting a polyol of the formula (I)
HO-A-OH (I)
where A is a carbon chain with about 2 to about 36 carbon atoms that is aliphatic linear or branched, saturated or unsaturated, or aromatic, or CH₂CH(OH)CH₂, CH₂C(CH₂ OH)₂CH₂, CH₂C(CH₂OH)(CH₃)CH₂, CH₂C(CH₂OH)(CH₂CH₃)CH₂, p-tetrahydrofuran, erythritol or an ester of di- or tricarboxylic acids with ethylene or propylene glycol, and wherein A can optionally be alkoxylated or reacted with hydroxy carboxylic acids, with an aminocarboxylic acid of formula II or its cyclic amide of the formula III 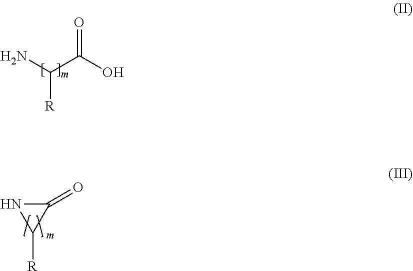
where m is an integer of 1 to 8 in formula II and 3 to 8 in formula III, each R independently is hydrogen or a C1-C4 alkyl group, or CH₂CH₂COOH, or CH₂COOH, or (CH₂)₄NH₂
in the presence of a Brønsted-Lowry acid at a temperature of between about 60 and about 200 degrees C. wherein the total molar amount of aminocarboxylic acid or its cyclic amide to the molar amount of the polyol is from about n:0.8 to about n:1.5 wherein n is the total number of hydroxyl groups on the polyol and wherein the Brønsted-Lowry acid is not added to the reaction mixture until at least about 50% of the total of the polyol and the aminocarboxylic acid or its cyclic amide are dosed.

2. Process of claim 1 wherein the total molar amount of aminocarboxylic acid or cyclic amide to the molar amount of the polyol is from about n:0.9 to about n:1.1.

3. Process of claim 1 wherein a cyclic amide of formula III is used wherein m is 3, 4, 5 or 6.

4. Process of claim 1 wherein the Brønsted-Lowry acid is sulfuric acid or phosphoric acid.

5. Process of claim 1 wherein the polyol is chosen from ethylene glycol, propylene glycol, butylene glycol, polyethylene glycol, polypropylene glycol, polybutylene glycol, glycerol, poly tetrahydrofuran, pentaerythritol, trimethylolpropane, trimethylolethane, diethylenesuccinate, dimeric fatty alcohol, 1,6-hexandiol, 1,4-butandiol, 1,8-octandiol, 1,10-decandiol, erythritol, optionally alkoxylated or reacted with hydroxy carboxylic acid, and combinations thereof.

6. Process of claim 1 wherein the polyol has a boiling point of up to about 220 degrees C., or, can form an azeotrope with water having a boiling point up to about 220 degrees C.

7. Process of claim 1 wherein the temperature is from about 60 to about 150 degrees C.

8. Process of claim 1 wherein the process is done with solvent in an amount of greater than about 0 to about 50 wt %, based on total reactants.

9. Process of claim 8 wherein the solvent is an organic solvent that is not aromatic and does not comprise halogens or alcohol units.

10. An amine-functional ester obtained from the process of claim 1.

11. The amine-functional ester of claim 10 having a structure of formula (IV) 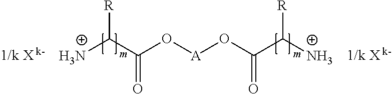
wherein k is a value of 1 to 3, X is an anion derivable from deprotonating the Brønsted-Lowry acid, m is an integer of 1 to 8, each R independently is hydrogen or a C1-C4 alkyl group, or CH₂CH₂COOH, or CH₂COOH, or (CH₂)₄NH₂, and wherein A is a carbon chain with about 2 to about 36 carbon atoms that is aliphatic linear or branched, saturated or unsaturated, or aromatic, or CH₂CH(OH)CH₂, CH₂C(CH₂ OH)₂CH₂, CH₂C(CH₂OH)(CH₃)CH₂, CH₂C(CH₂OH)(CH₂CH₃)CH₂, p-tetrahydrofuran, erythritol or an ester of di- or tricarboxylic acids with ethylene or propylene glycol, and wherein A can optionally be alkoxylated or reacted with hydroxy carboxylic acids.

12. The amine-functional ester of claim 10 wherein X is sulfate or hydrogen sulfate.

13. The amine-functional ester of claim 10 comprising between about 0 and about 30 mole % on the basis of total moles of compounds (IV) of compounds of formula (VI) 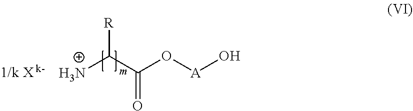
wherein k is a value of 1 to 3, X is an anion derivable from deprotonating the Brønsted-Lowry acid, m is an integer of 1 to 8, each R independently is hydrogen or a C1-C4 alkyl group, or CH₂CH₂COOH, or CH₂COOH, or (CH₂)₄NH₂, and wherein A is a carbon chain with about 2 to about 36 carbon atoms that is aliphatic linear or branched, saturated or unsaturated, or aromatic, or CH₂CH(OH)CH₂, CH₂C(CH₂ OH)₂CH₂, CH₂C(CH₂OH)(CH₃)CH₂, CH₂C(CH₂OH)(CH₂CH₃)CH₂, p-tetrahydrofuran, erythritol or an ester of di- or tricarboxylic acids with ethylene or propylene glycol, and wherein A can optionally be alkoxylated or reacted with hydroxy carboxylic acids.

14. The process of claim 2 wherein the polyol is chosen from ethylene glycol, propylene glycol, butylene glycol, polyethylene glycol, polypropylene glycol, polybutylene glycol, glycerol, poly tetrahydrofuran, pentaerythritol, trimethylolpropane, trimethylolethane, diethylenesuccinate, dimeric fatty alcohol, 1,6-hexandiol, 1,4-butandiol, 1,8-octandiol, 1,10-decandiol, erythritol, optionally alkoxylated or reacted with hydroxy carboxylic acid, and combinations thereof.

15. The process of claim 3 wherein the polyol is chosen from ethylene glycol, propylene glycol, butylene glycol, polyethylene glycol, polypropylene glycol, polybutylene glycol, glycerol, poly tetrahydrofuran, pentaerythritol, trimethylolpropane, trimethylolethane, diethylenesuccinate, dimeric fatty alcohol, 1,6-hexandiol, 1,4-butandiol, 1,8-octandiol, 1,10-decandiol, erythritol, optionally alkoxylated or reacted with hydroxy carboxylic acid, and combinations thereof.

16. The process of claim 4 wherein the polyol is chosen from ethylene glycol, propylene glycol, butylene glycol, polyethylene glycol, polypropylene glycol, polybutylene glycol, glycerol, poly tetrahydrofuran, pentaerythritol, trimethylolpropane, trimethylolethane, diethylenesuccinate, dimeric fatty alcohol, 1,6-hexandiol, 1,4-butandiol, 1,8-octandiol, 1,10-decandiol, erythritol, optionally alkoxylated or reacted with hydroxy carboxylic acid, and combinations thereof.

17. Process of claim 1 wherein the process is done without solvent.
